(19) United States
(12) Patent Application Publication
(10) Pub. No.: US 2004/0243144 A1
(43) Pub. Date: Dec. 2, 2004

(54) Device

(76) Inventors: Frank Bonadio, Bray (IE); John Butler, Dublin (IE); Shane Joseph MacNally, County Wicklow (IE)

Correspondence Address: JACOBSON HOLMAN PLLC, 400 SEVENTH STREET N.W., SUITE 600, WASHINGTON, DC, 20004, US

(21) Appl. No.: 10/859,168

(22) Filed: Jun. 3, 2004

Related U.S. Application Data
(63) Continuation of application No. PCT/IE02/00165, filed on Dec. 4, 2002.

(30) Foreign Application Priority Data
Dec. 4, 2001 (IE) 2001/1037
Dec. 4, 2001 (IE) 2001/1038

(51) Int. Cl. A61F 11/00

(52) U.S. Cl. 606/108

(57) ABSTRACT

A guide device for assisting advancement of a probe though a passageway, such as the colon, by maintaining the probe spaced from the interior walls of the passageway during advancement. The guide (1) comprises an evertable tube (3) having longitudinally extending outer and inner walls (2, 4) which are connected at each end to define an enclosed inflation space (5) therebetween. The inner walls (4) define a lumen (8) and the tube is inflatable to grip a probe (7) in the lumen such that the tube is evertable with advancement of a probe (7). A skeletal member (6) extends longitudinally in the inflation space. In particular this invention relates to a guide device which facilitates enhanced vision during probe advancement, especially in a tortuous passageway such as the colon.

### INTRODUCTION

This invention relates to a guide device for assisting advancement of a probe through a passageway by maintaining the probe spaced from the interior walls of the passageway during advancement of the probe through tie passageway. In particular this invention relates to a guide device which facilitates enhanced vision during probe advancement, especially in a tortuous passageway such as the colon.

It is known to use an everting tube to advance a probe through a passageway. For example, U.S. Pat. No. 4,321,915 describes such a flexible, everting tube. By applying a fluid pressure to the tube a fibre optic tool extending through the tube is gripped and pulled along by the tube as it everts. One problem with known guide devices of this type is that because of the tube eversion action, a tool extending through the tube advances at twice the rate of the tube. Thus, the tip of the tool extends beyond the leading edge of the everting tube. U.S. Pat. No. 4,321,915 describes applying a suction to the tube when the tip of the tool has extended a distance beyond the leading edge of the tube. The suction causes the tube to disengage from the tool and allows an operator to manually retract the tool into the tube.

This procedure is generally inconvenient and inefficient, especially when navigating tortuous passageways such as the colon.

This invention is aimed at providing a guide device which will address at least some of these problems.

### STATEMENTS OF INVENTION

According to the invention there is provided a guide device for a probe, the device comprising:

an evertable tube,

the tube having longitudinally extending outer and inner walls which are connected at each end to define an enclosed inflation space therebetween,

the inner wall defining a lumen and the tube being inflatable to grip a probe in the lumen such that the tube is evertable with advancement of a probe.

In one case a skeletal member extends longitudinally in the inflation space.

The skeletal member may comprise a tubular skeletal member.

In one case the skeletal member is at least partially corrugated.

In another case the skeletal member may be slitted. The slit may extend at least partially in a spiral.

In one embodiment the guide device may comprise a skeletal member longitudinally in the expansion space of the tube.

In another embodiment the, tube of the guide device may be longitudinally extendable over a probe in the lumen for alignment of an end of the probe with an end of the tube.

In a further embodiment the guide device may comprise a stop to selectively prevent tube eversion.

According to another aspect of the invention, there is provided a guide device for a probe, the device comprising:

an evertable tube with a lumen therethrough, the tube being inflatable to grip a probe in the lumen such that the tube everts with advancement of the probe, the tube being advanceable over a probe in the lumen to align an end of the tube with an end of the probe.

In one case the outer wall of the tube is connected at each end to the inner wall of the tube to define an enclosed inflation space therebetween.

In one embodiment of the invention the tube is longitudinally extendable from a shortened configuration to a lengthened configuration over a probe in the lumen to align an end of the tube with an end of the probe. In the shortened configuration a part of the tube may be bunched. The bunched part of the tube is provided on the outer wall of the tube.

At least part of the tube may be inflatable to longitudinally extend the tube. The inflatable part of the tube may be conically shaped.

The device may comprise stop means to selectively prevent longitudinal extension of the tube.

In another case the device comprises stop means to selectively prevent tube eversion. The stop means may comprise a clamp engagable with the tube.

In another embodiment of the invention the device comprises a skeletal member extending longitudinally of the tube. The tube may have an inflation space and the skeletal member extends in the inflation space.

In another aspect of the invention, there is provided a guide device for a probe; the device comprising:

an evertable tube with a lumen therethrough, the tube being inflatable to grip a probe in the lumen such that the tube everts with advancement of the probe; and

a skeletal member extending longitudinally of the tube.

The skeletal member is preferably tubular and may extend co-axially around the lumen.

The skeletal member may be at least partially corrugated. This provides kink resistance. In one case the corrugation extends in a convoluted manner.

In another case the corrugation extends in a loop.

In one embodiment the skeletal member has at least one slit which may extend at least partially in a spiral.

In a further aspect of the invention, there is provided a probe system comprising a probe, and a guide device of the invention for the probe.

According to another aspect of the invention, there is provided a method of advancing a probe through a passageway, the method comprising the steps of:

providing a probe;

providing a guide device for the probe, the guide device having a lumen therethrough;

inserting the probe through the lumen of the guide device;

inflating the guide device to grip the probe;

advancing the probe through the passageway with an associated eversion of the guide device;

advancing at least part of the guide device over the probe to align the leading end of the guide device with the leading end of the probe.

In one embodiment the method comprises the step of deflating the guide device and the guide device is then advanced over the probe by pushing the guide device over the probe.

In another embodiment at least part of the guide device is advanced over the probe by inflating at least part of the guide device. Ideally a leading end part of the guide device is inflated. Most preferably part of the guide device is deflated before inflation of the leadings end part.

In another case the method comprises the step of releasing a stop means before advancing at least part of the guide device over the probe.

The probe is preferably advanced through the passageway by pushing the probe through the passageway.

In another embodiment the probe is maintained in a fixed position during the step of advancing at least part of the guide device over the probe.

Preferably the method comprises the step of releasing a stop means before eversion of the guide device.

At least some of the steps may be repeated to advance the probe in an incremental manner through the passageway.

According to another aspect of the invention there is provided a method of advancing a probe through a passageway, the method comprising the steps of:

providing a probe;

providing a guide device for the probe, the guide device comprising an evertable tube having longitudinally extending outer and inner walls which are connected at each end to define an enclosed inflation space therebetween, the inner wall defining a lumen;

inserting the probe through the lumen of the guide device;

inflating the guide device to grip the probe;

pushing the probe to advance the probe through the passageway which causes eversion of the guide device;

moving at least part of the probe relative to the guide device to align the leading end of the probe with the leading end of the guide device.

In one embodiment at least part of the guide device is advanced over the probe to align the leading end of the guide device with the leading end of the probe.

In another embodiment the probe is retracted through the lumen to align the leading end of the guide device with the leading end of the probe.

According to a further aspect of the invention there is provided a method of bridging a tortuous section of a passageway such as a sigmoid colon comprising the steps of:

providing an evertable tube, the tube having longitudinally extending outer and inner walls which are connected at each end to define an enclosed space, the inner wall defining a lumen;

providing a skeletal member extending longitudinally in the enclosed space of the evertable tube;

providing a delivery member;

inserting the delivery member through the lumen of the evertable tube;

inflating the tube to grip the delivery member;

advancing the delivery member through the passageway with associated eversion of the tube and advancement of the skeletal member; and

deflating the evertable tube such that the skeletal member bridges a section of the passageway.

In one embodiment the method comprises the step of withdrawing the delivery member after bridging of the section of the passageway.

In another embodiment the method comprises the step of insertion of a probe in a lumen defined by the skeletal member.

### BRIEF DESCRIPTION OF THE DRAWINGS

The invention will be more clearly understood from the following description of some embodiments thereof, given by way of example only, with reference to the accompanying drawings, in which:

FIG. 1 is a perspective view of a guide device according to the invention in a deflated configuration;

FIG. 2 is a perspective view of the guide device of FIG. 1 in an inflated configuration;

FIG. 3 is a side, cross-sectional view of the guide device of FIG. 1 in the deflated configuration;

FIG. 4 is a side, cross-sectional view of the guide device of FIG. 1 in the inflated configuration;

FIGS. 5 to 8 are views similar to FIGS. 1 to 4 of a probe extending through a lumen of the guide device of FIG. 1;

FIG. 9 is a side, partially cross-sectional view of a skeletal member of the guide device of FIG. 1;

FIGS. 10 and 11 are side views of other skeletal members;

FIGS. 12 to 20 are side, partially cross-sectional views illustrating advancement of the probe of FIGS. 5 to 8 through a passageway using the guide device of FIG. 1;

FIGS. 21 to 28 are perspective views illustrating schematically advancement of the probe of FIGS. 5 to 8 through a curved passageway using the guide device of FIG. 1;

FIG. 29 is a side, partially cross-sectional view of another guide device according to the invention with a probe extending through a lumen of the guide device;

FIGS. 30 to 33 are side, partially cross-sectional views illustrating schematically advancement of the probe of FIG. 29 through a passageway using the guide device of FIG. 29;

FIG. 34 is a side, partially cross-sectional view of another guide device according to the invention with a probe extending through a lumen of the guide device;

FIGS. 35 and 36 are side, partially cross-sectional views illustrating schematically advancement of the probe of FIG. 34 through a passageway using the guide device of FIG. 34;

FIG. 37 is a perspective view of a leading end of another guide device according to the invention with a probe extending through a lumen of the guide device; and

FIGS. 38 to 41 are side, partially cross-sectional views illustrating schematically advancement of the probe of FIG. 37 through a passageway using the guide device of FIG. 37.

### DETAILED DESCRIPTION

Referring to the drawings there is illustrated a guide device 1 according to the invention. The guide device 1 is suitable for assisting advancement of a probe 7, such as a colonoscope, through a passageway, such as a body cavity 11, for example the colon.

The device 1 comprises an evertable tube 3 with a central lumen 8 therethrough. The tube 3 can be inflated to grip the probe 7 in the lumen 8 such that the tube 3 will evert with advancement of the probe 7. The device 1 further comprises means to facilitate advancing at least part of the tube 3 over the probe 7 in the lumen 8 to align an end of the tube 3 with an end of the probe 7.

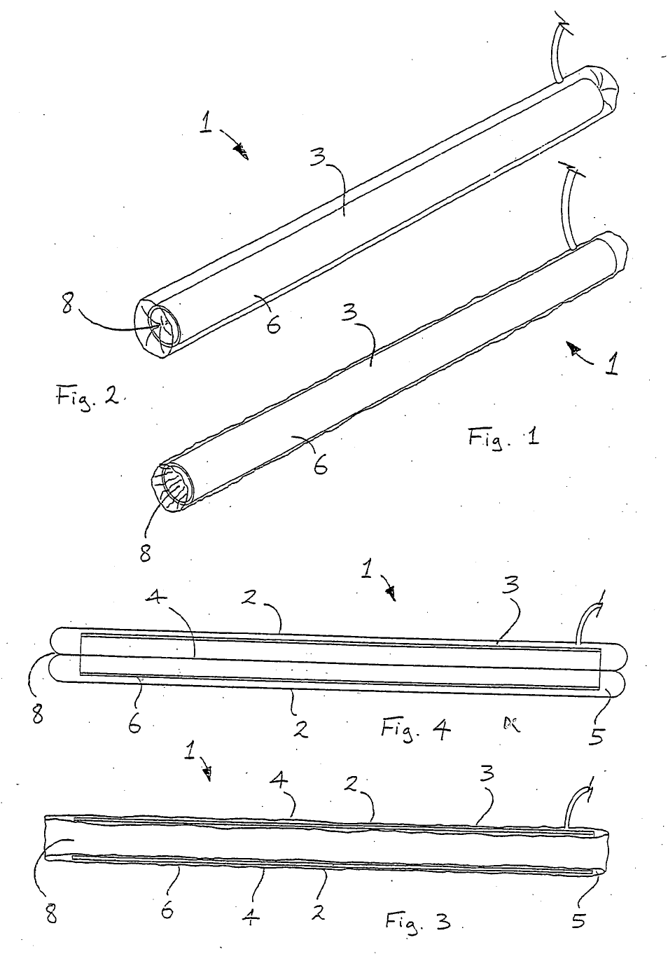

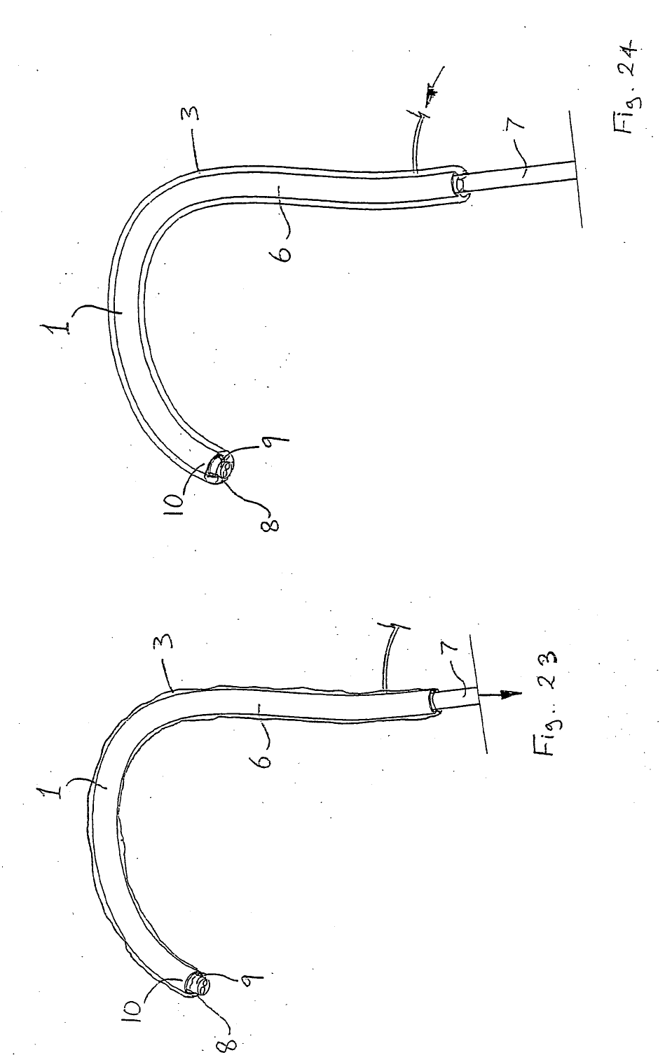

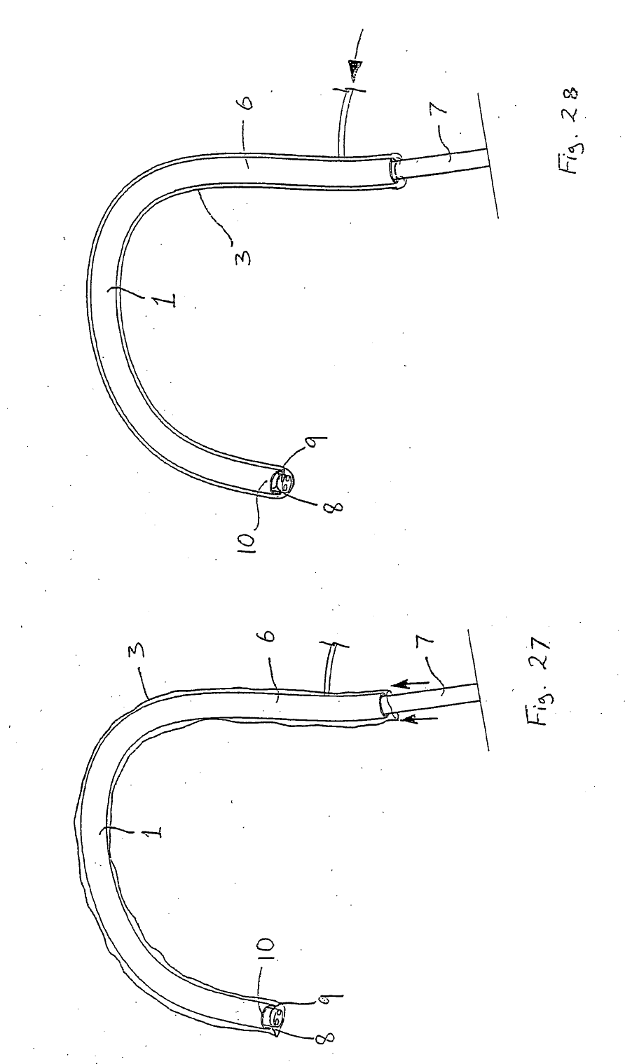

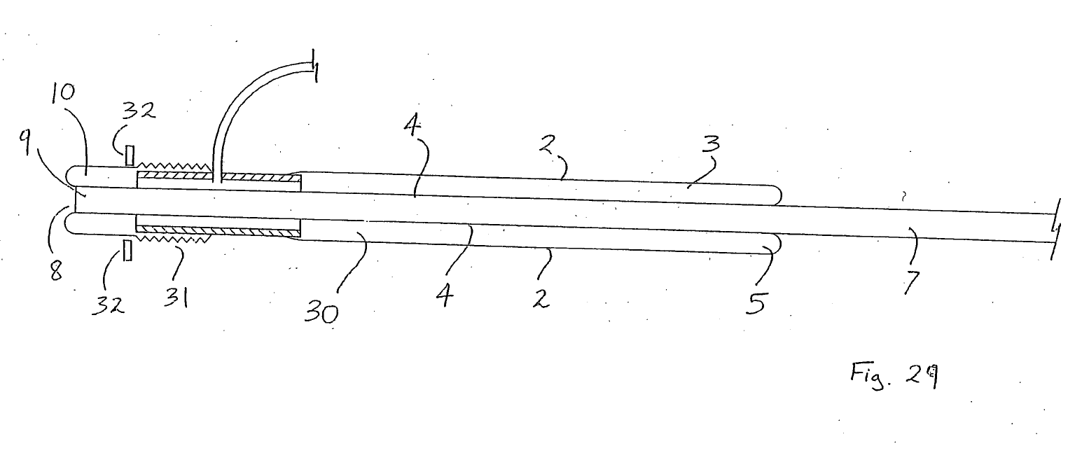

FIGS. 1 to 28 illustrate a particular embodiment of the device 1.

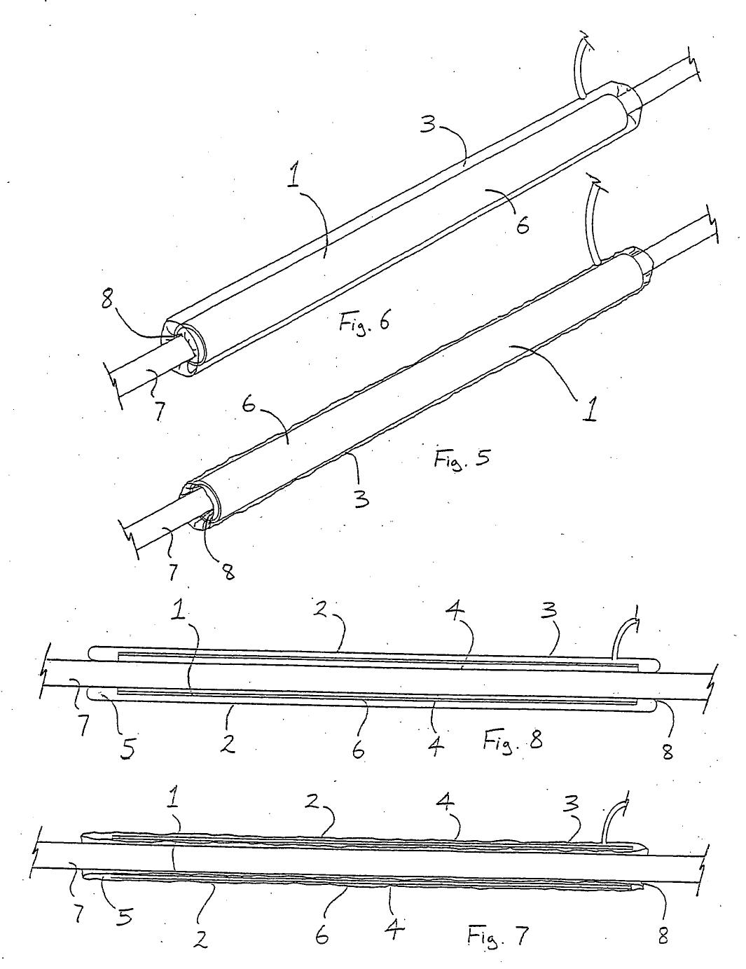

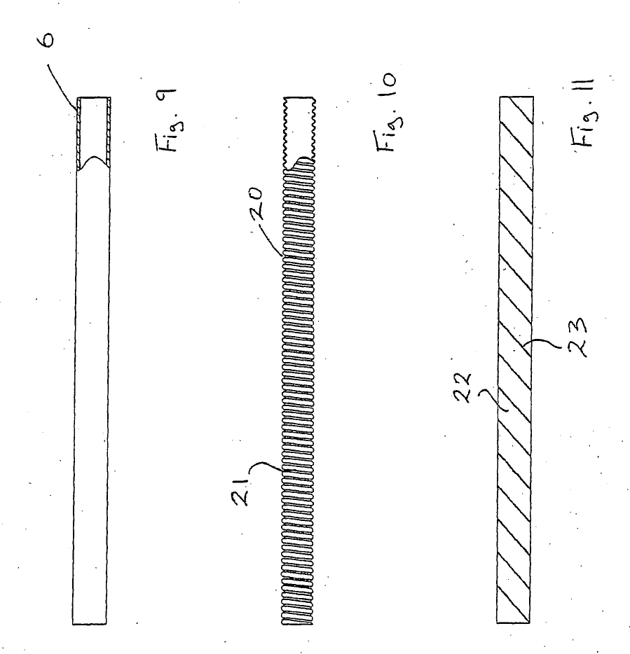

As illustrated in FIGS. 3 and 4, an outer wall 2 of the evertable tube 3 is connected at each end to an inner wall 4 of the tube 3, such that an enclosed inflation space 5 is defined between the walls 2, 4. The tube 3 may be inflated by air or using any suitable fluid.

In this case, the device 1 has a tubular skeletal member 6, as illustrated in FIG. 9. The skeletal member may longitudinally stiffen the flexible tube 3. In this way, the stiffened device 1 may be advanced over a probe 7 in the lumen 8 or vice versa when the tube 3 is deflated. The skeletal member 6 is located within the inflation space 5, and extends co-axially around the lumen 8.

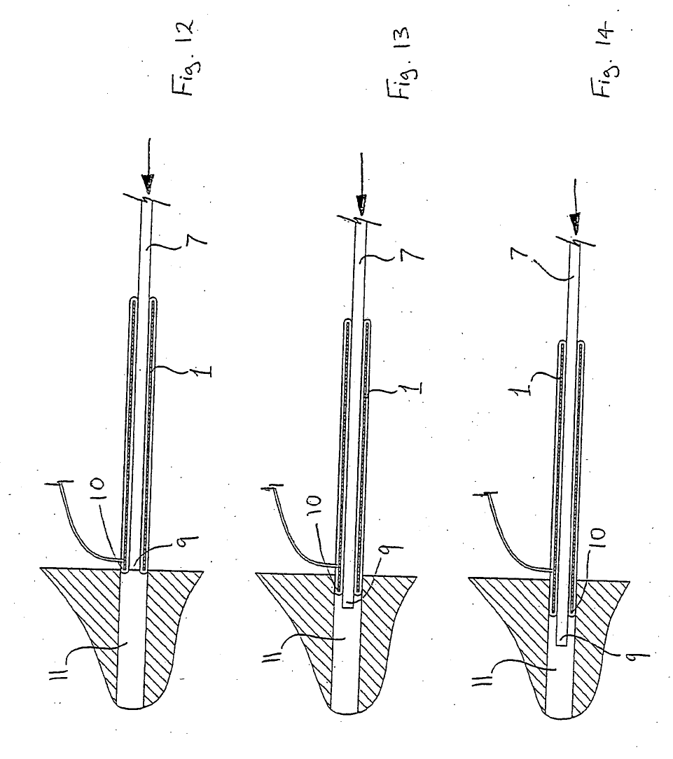

In use, the probe 7 is inserted through the lumen 8 of the deflated tube 3 until the leading or distal end 9 of the probe 7 is aligned with the leading or distal end 10 of the guide device 1. The tube 3 is then inflated to grip the probe 7, and the probe 7 and guide device 1 are now ready for insertion into the passageway 11 (FIG. 12).

The probe 7 is advanced through the passageway 11 by pushing the probe 7 distally. Because the inflated tube 3 grips the probe 7, the tube 3 everts as the probe 7 advances distally through the passageway 11 (FIGS. 13 and 14).

The inflated tube 3 acts as a spacing means to prevent the probe 7 from engaging against the interior walls of the passageway 11 as the probe 7 advances through the passageway 11. In this manner, the probe 7 advances through the passageway 11 with a frictionless rolling action of the guide device 1 and with substantially no frictional contact between the probe 7 and the passageway 11.

As illustrated in FIGS. 12 to 14, due to the everting action of the guide device 1 with the probe 7, the leading end 9 of the probe 7 travels twice the distance of the leading end 10 of the guide device 1. Thus, the probe leading end 9 projects distally from the guide device 1.

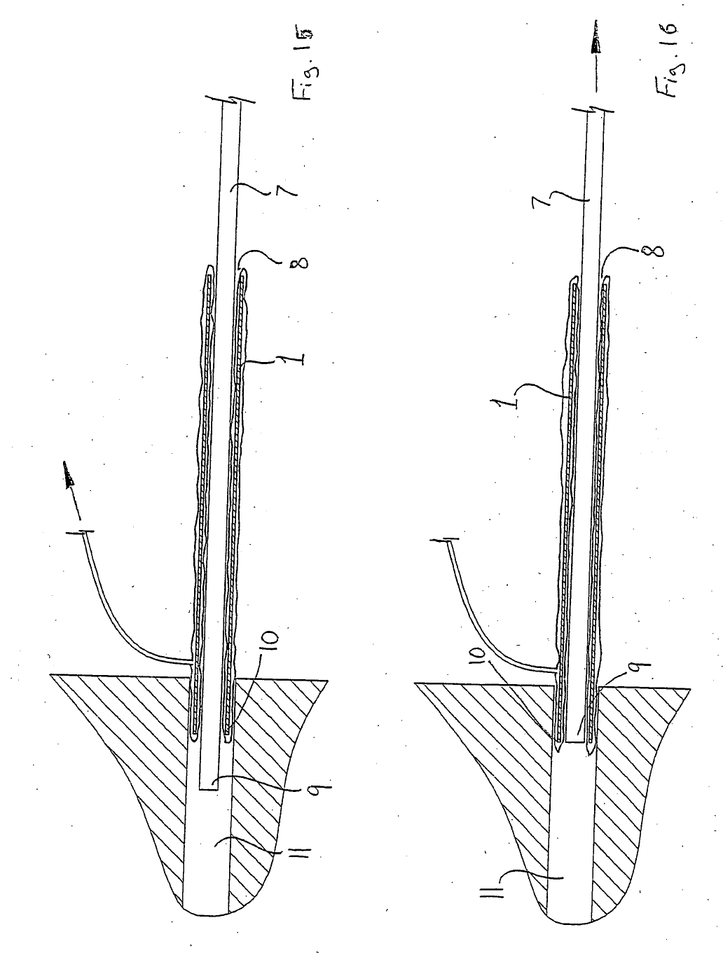

To realign the two leading ends 9, 10, the tube 3 is deflated so that the probe 7 is no longer gripped by the tube 3 (FIG. 15), but there is still a central skeletal member 6 within the deflated tube 3. This allows the probe 7 to be retracted through the lumen 8 of the tube 3 until the leading ends 9, 10 are aligned (FIG. 16).

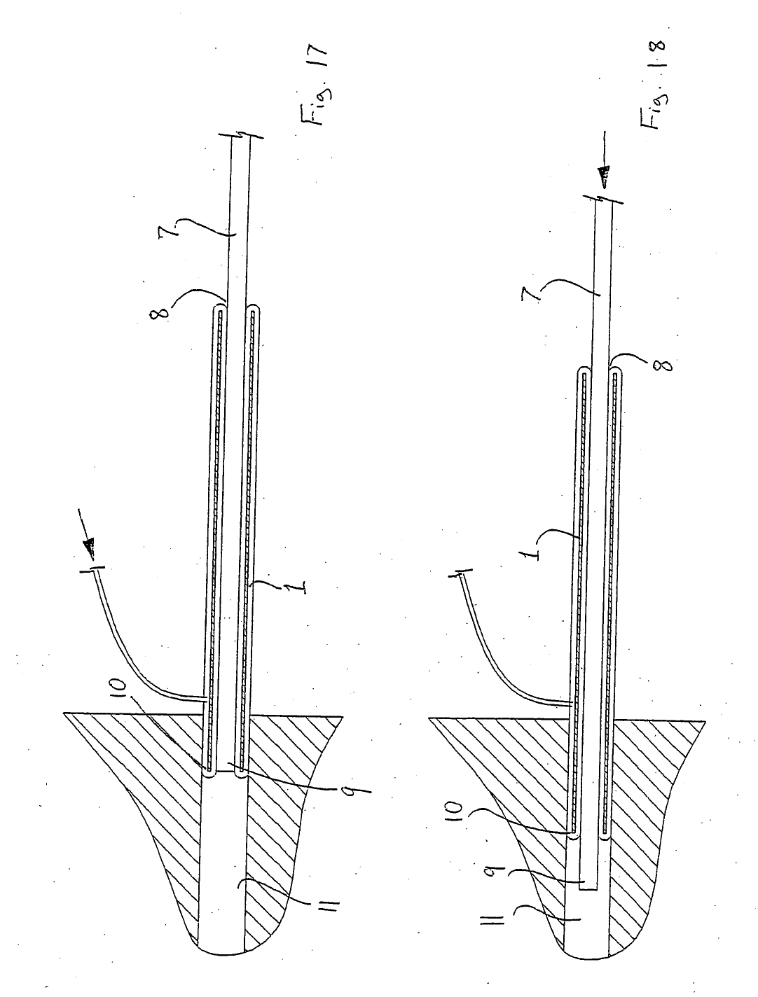

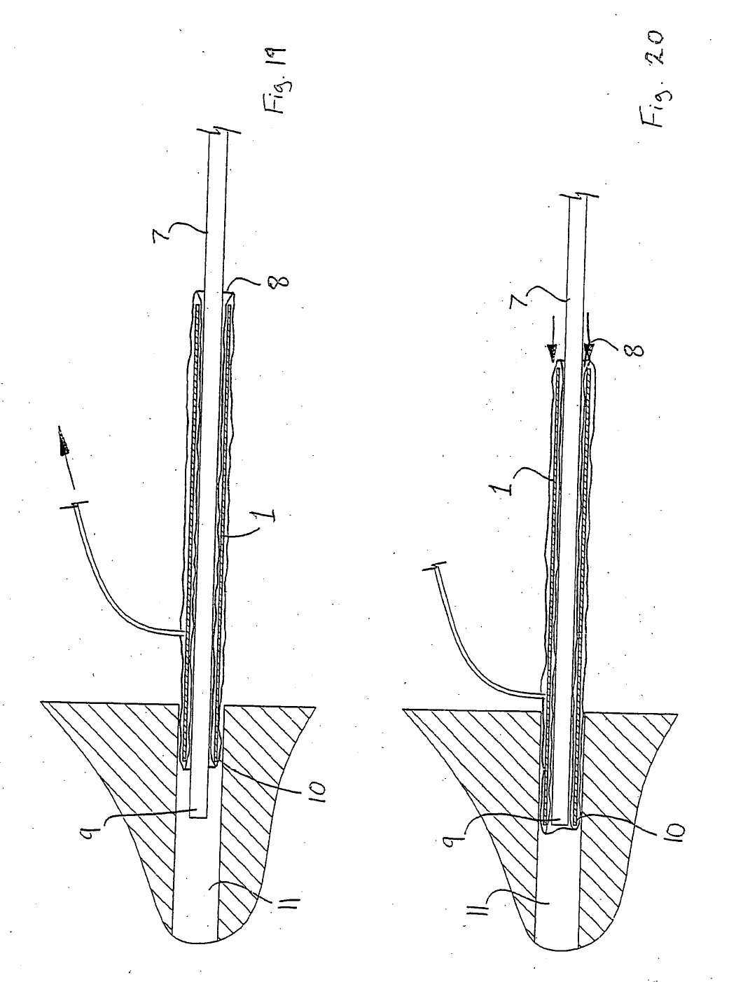

The tube 3 is re-inflated (FIG. 17), and the probe 7 is further advanced through the passageway 11 by pushing the probe 7 distally (FIG. 18).

The steps described above with reference to FIGS. 15 to 18 may be repeated as desired by the user to advance the probe 7 through the passageway 11 in an incremental manner.

In this way, the probe 7 advances through the passageway 11 in a “2 steps forward-1 step back” manner.

It will be appreciated that the guide device 1 may alternatively be advanced over the probe 7 when the tube 3 is deflated to align the leading end 10 of the guide device 1 with the leading end 9 of the probe 7, as illustrated in FIGS. 19 and 20. In this case, the probe 7 is maintained in a fixed position during realignment of the leading ends 9, 10.

Realignment of the leading ends 9, 10 by advancing the guide device, 1 over the probe 7 has the advantage that all movement of the guide device 1 and the probe 7 is in the distal direction through the passageway 11. This provides for a smooth advancement procedure, which can be of particular importance when the probe, is being advanced through a floppy or flexible passageway, such as a colon, which has regions of tortuosity.

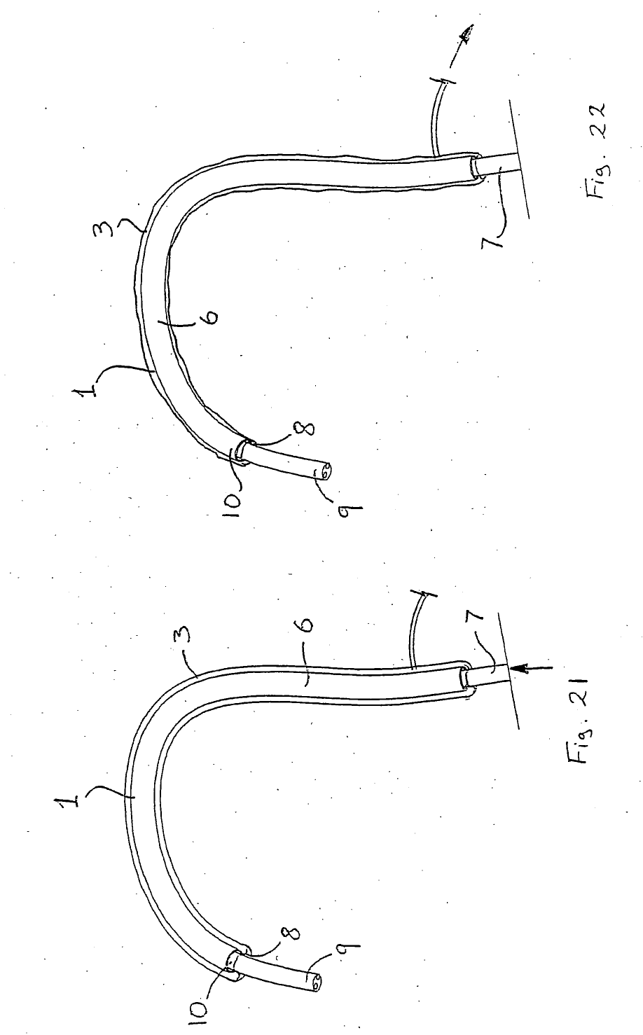

The guide device 1 is suitable for assisting the advancement of the probe 7 through curved or tortuous passageways such as illustrated in FIGS. 21 to 28.

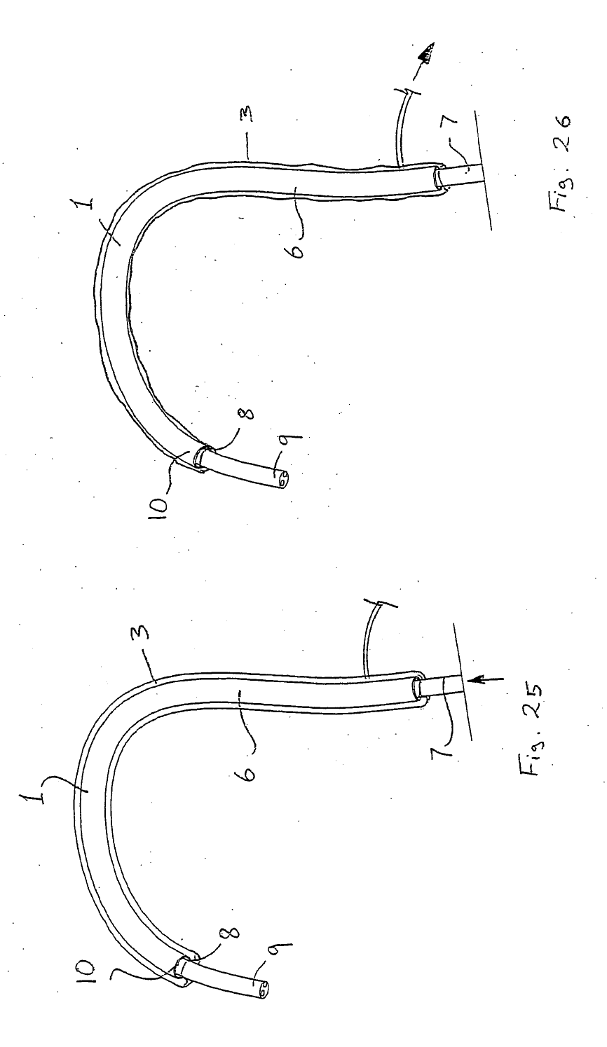

FIGS. 21 to 28 illustrate advancement of the probe 7 through a curved passageway such as a sigmoid colon. The leading end 9 of the probe 7 can be aligned with the leading end 10 of the guide device 1 by retracting the probe 7 through the lumen 8 of the deflated tube 3 (FIGS. 21 to 24), or by advancing the deflated guide device 1 over the probe 7 (FIGS. 25 to 28), in a manner similar to that described previously with reference to FIGS. 12 to 20.

The skeletal member 6 may be a simple tube as illustrated in FIG. 9. Alternatively the skeletal member 21 may comprise at least one corrugation 20 on the skeletal member 21 to resist kinking of the skeletal member 21 as the guide device 1 bends around a curve in the passageway. The corrugation 20 may extend along the skeletal member 21 in a convoluted manner, as illustrated in the skeletal member 21 of FIG. 10, or the corrugation may extend around the skeletal member 21 in a loop.

A further alternative is illustrated in FIG. 11, in which a skeletal member 22 has a slit 23 through the wall, the slit extending along the skeletal member 22 in a spiral. The slit skeletal member 22 is normally flexible such that when the tube 3 is in the inflated configuration, the skeletal member 22 provides minimum resistance to eversion of the tube 3 and minimum resistance to bending of the guide device 1 through the passageway. However, when the tube 3 is deflated it has been found that the skeletal member 22 becomes much stiffer, and thus it is relatively easy to manipulate the guide device 1 and the probe 7 to align the leading ends 9, 10, as described above.

In the embodiments described above the probe is retracted into the tube. It is also possible, as an alternative, to advance the tube over the exposed tip of the probe. This can be achieved in a number of ways. For example, in the embodiments described above the skeletal member may facilitate advancement of the tube over the probe. Alternatively the tube may be lengthened. For example, the inner lumen of the everting tube may be maintained stationary relative to the probe while the outer wall of the tube is advanced over the probe.

FIGS. 29 to 33 illustrate another guide device 30, which is similar to the guide device 1 of FIGS. 1 to 28, and similar elements in FIGS. 29 to 33 are assigned the same reference numerals.

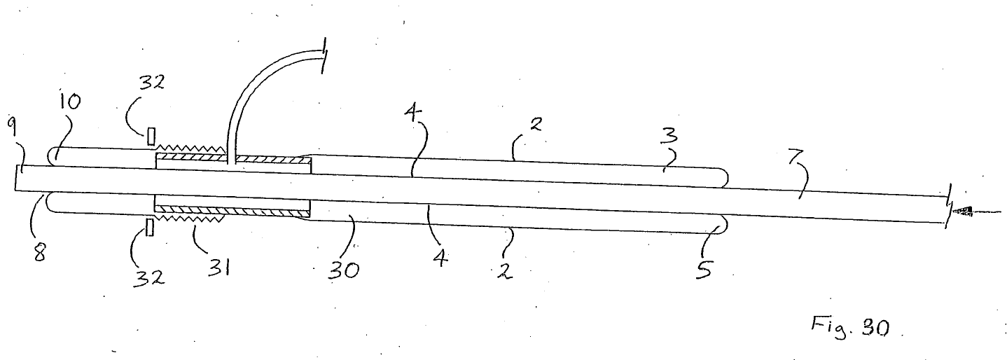

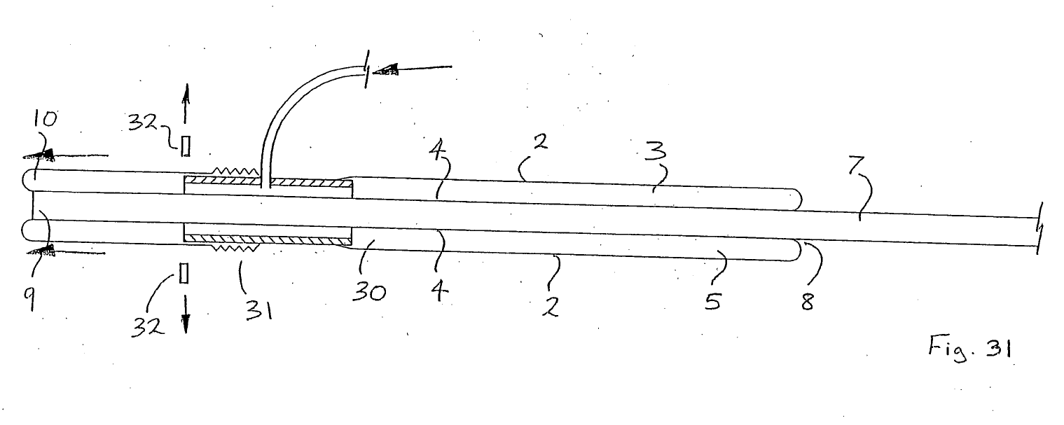

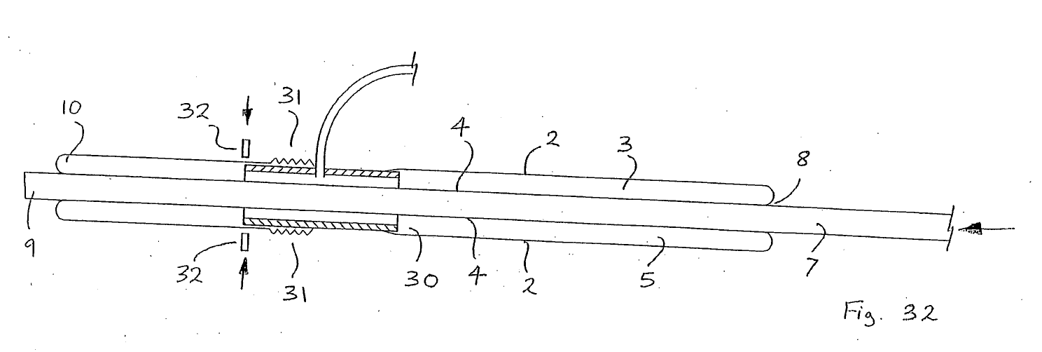

In this case, the tube 3 is longitudinally extendable from a shortened configuration (FIG. 30) to a lengthened configuration (FIG. 31) by inflation to advance part of the tube 3 over the probe 7 in the lumen 8 to align the leading end 10 of the guide device 30 with the leading end 9 of the probe 7.

In the shortened configuration, part of the outer wall 2 of the tube 3 is bunched 31. A stop means, in this case in the form of a clamp 32, is provided to selectively prevent longitudinal extension of the tube 3.

In use, the probe 7 is inserted through the lumen 8 of the deflated tube 3 until the leading end 9 of the probe 7 is aligned with the leading end 10 of the guide device 30. The tube 3 is then inflated to grip the probe 7. In this inflated configuration, the clamp 32 engages the outer wall 2 of the tube 3 distally of the bunching 31 to initially prevent longitudinal extension of the tube 3 (FIG. 29).

The probe 7 is advanced through the passageway by pushing the probe 7 distally which causes eversion of the guide device 30 (FIG. 30). To realign the two leading ends 9, 10, the clamp 32 is moved outwardly to release the bunching 31, and the tube 3 is further inflated to advance part of the tube 3 over the probe 7 to align the leading end 10 of the guide device 30 with the leading end 9 of the probe 7 (FIG. 31).

When the leading ends 9, 10 have been aligned, the clamp 32 is moved inwardly to engage the outer wall 2 of the tube 3, and the probe 7 is further advanced through the passageway by pushing the probe 7 distally (FIG. 32).

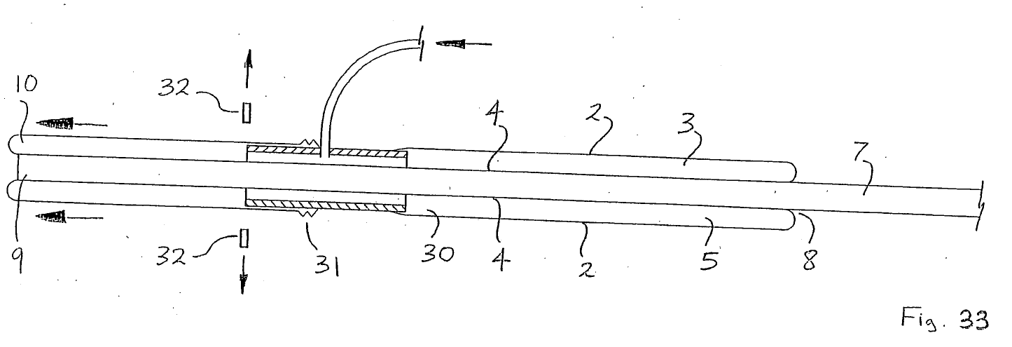

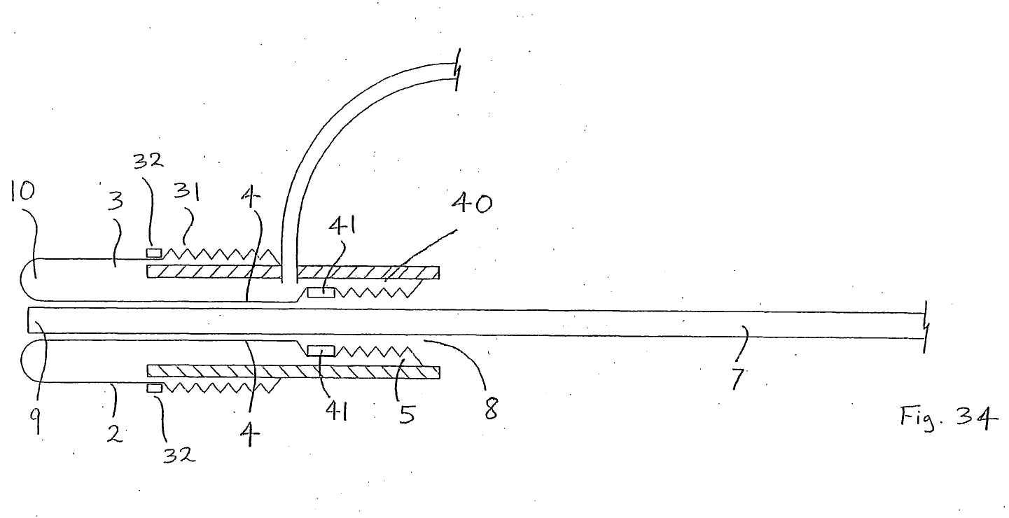

These steps to advance the probe 7 and realign the leading ends 9, 10 may be repeated (FIG. 33) as desired by the user to advance the probe 7 through the passageway in an incremental manner.

All movement of the guide device 30 and the probe 7 is in the distal direction through the passageway.

FIGS. 34 to 36 illustrate another guide device 40, which is similar to the guide device 30 of FIGS. 29 to 33, and similar elements in FIGS. 34 to 36 are assigned the same reference numerals.

In this case, the guide device 40 comprises an inner clamp 41 to selectively prevent eversion of the tube 3. When the clamp 41 engages the inner wall 4 of the tube 3 (FIG. 34) the inner wall 4 is prevented from moving distally, and thus the tube 3 is prevented from everting and the probe 7 is prevented from advancing through the passageway.

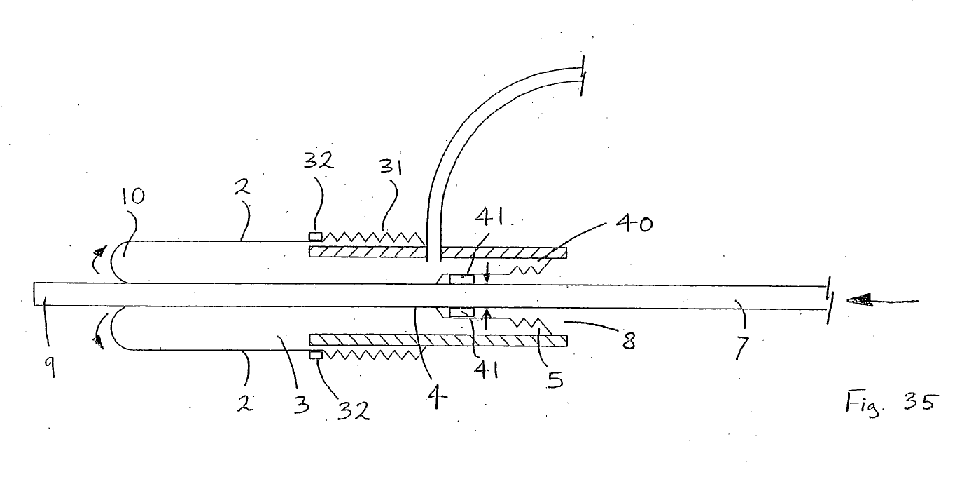

By moving the clamp 41 inwardly to release the inner wall 4 of the tube 3, the inner wall 4 can then move distally, and the tube 3 can evert as the probe 7 advances through the passageway (FIG. 35).

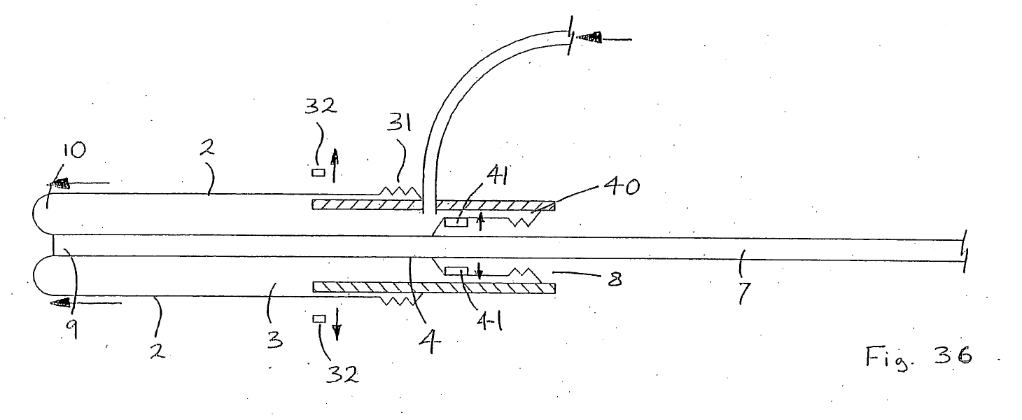

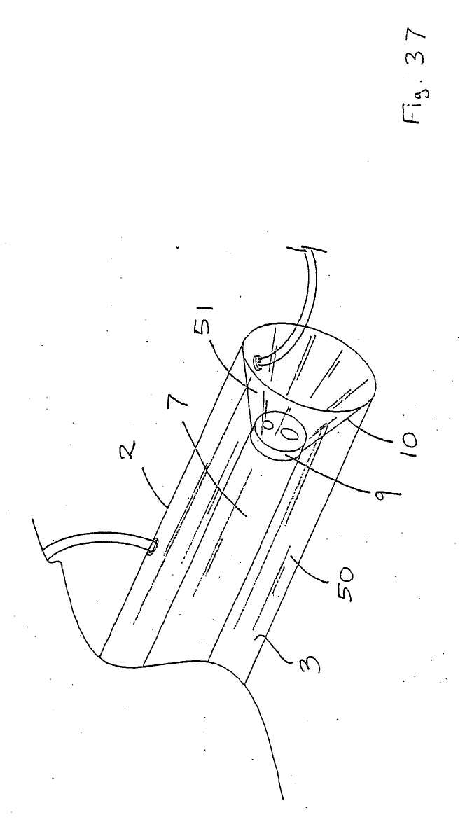

To realign the leading ends 9, 10, the clamp 41 is moved outwardly to engage the inner wall 4 of the tube 3. The outer clamp 32 is also moved outwardly and the tube 3 is inflated (FIG. 36), in a manner similar to that described previously with reference to FIG. 31.

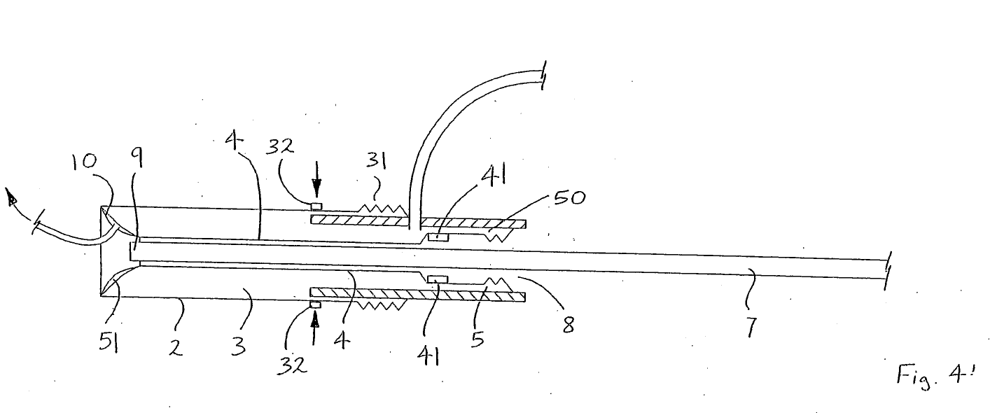

Referring to FIGS. 37 to 41, there is illustrated another guide device 50, which is similar to the guide device 40 of FIGS. 34 to 36, and similar elements in FIGS. 37 to 41 are assigned the same reference numerals.

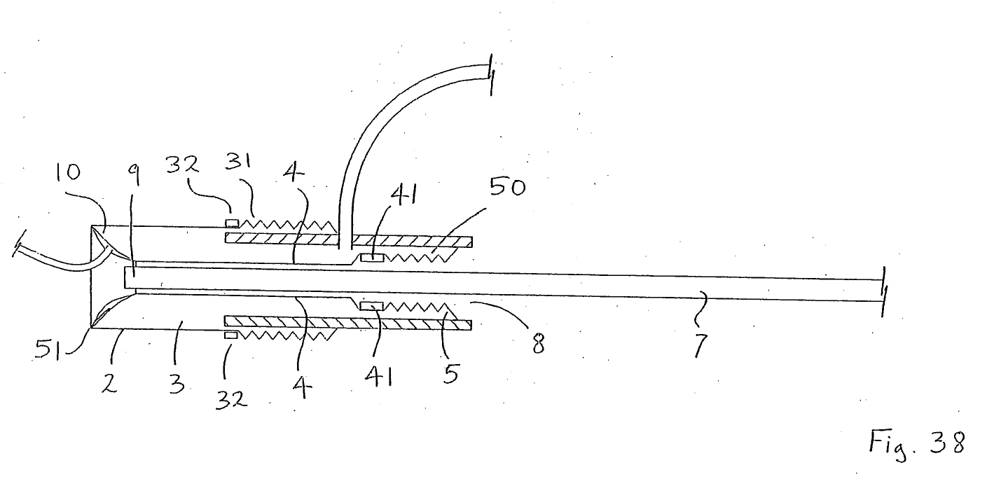

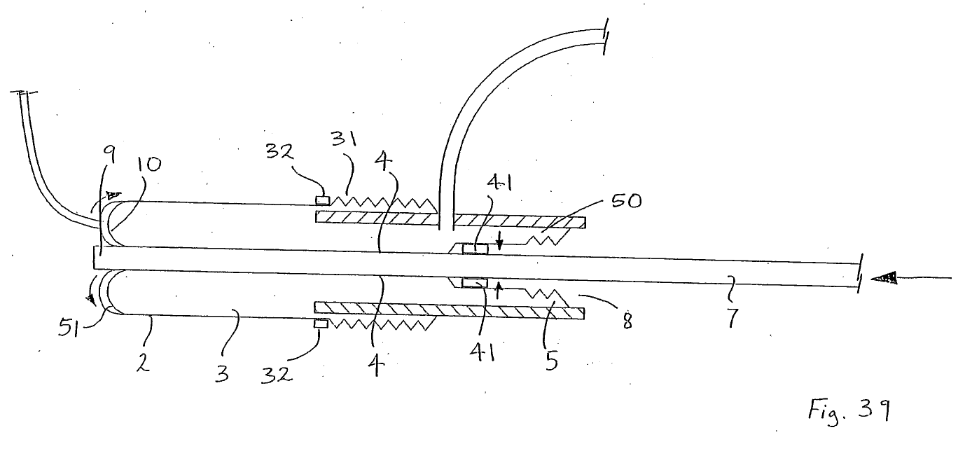

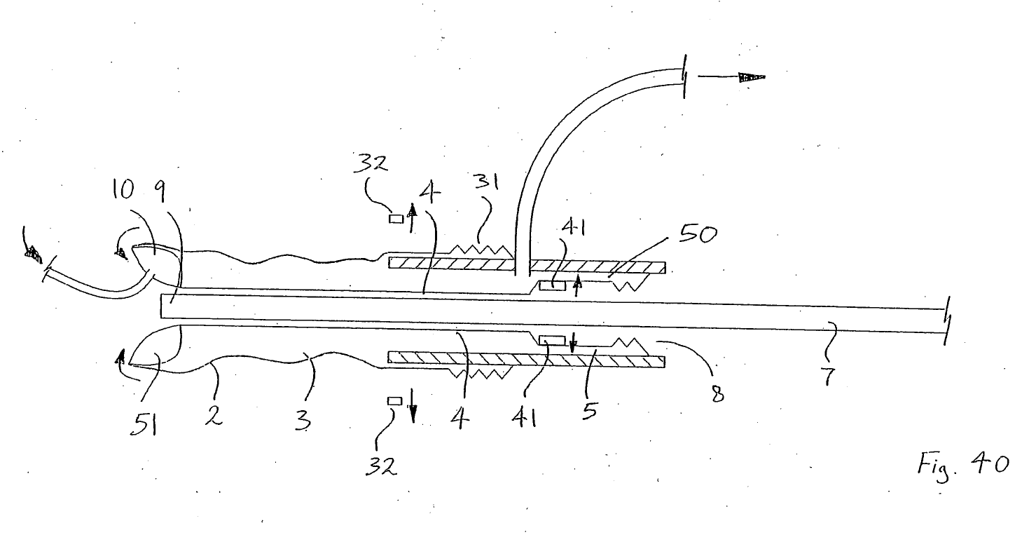

In this case, the tube 3 has an inflatable conical tip 51 at the leading end 10 of the guide device 50 (FIG. 37). The conical tip 51 is separated from the inflation space 5, as illustrated in FIG. 38, so that the conical tip 51 may be inflated and deflated independently of the inflation space 5 of the remainder of the tube 3.

In use, the probe 7 is inserted through the lumen 8 of the deflated tube 3 until the leading end 9 of the probe 7 is aligned with the leading end 10 of the guide device 1 (FIG. 38). The inflation space 5 of the tube 3 is then inflated to grip the probe 7. In this inflated configuration, the conical tip 51 remains deflated, and the clamp 32 engages the outer wall 2 of the tube 3 distally of the bunching 31 to initially prevent longitudinal extension of the tube 3.

The clamp 41 is moved inwardly to release the inner wall 4 of the tube 3, and the probe 7 is advanced through the passageway by pushing the probe 7 distally, which causes eversion of the guide device 50 (FIG. 39). In this everted configuration, the deflated tip 51 is bent proximally at the leading end 10 of the guide device 50.

To realign the leading ends 9, 10, the inner clamp 41 is moved outwardly to engage the inner wall 4 of the tube 3, and the outer clamp 32 is moved outwardly to release the bunching 31. The inflation space 5 of the tube 3 is then deflated, and the tip 51 is inflated which causes the tip 51 to flip over towards the leading end 9 of the probe 7. As the tip 51 flips over, the tip 51 pulls part of the tube 3 over the probe 7 to align the leading end 10 of the guide device 50 with the leading end 9 of the probe 7.

During realignment of the leading ends 9, 10, the main body of the tube 3 is deflated. In this way, contact between the tube 3 and the interior walls of the passageway is minimised during advancement of the tube 3 over the probe 7. This provides for a smooth, efficient realignment action.

When the leading ends 9, 10 have been realigned, the clamp 32 is moved inwardly to engage the outer wall 2 of the tube 3, and the conical tip 51 is deflated (FIG. 41). The probe 7 is now ready for further advancement through the passageway by repeating the steps described above with reference to FIGS. 37 to 41.

It will be appreciated that the invention also provides a method and an apparatus for bridging a tortuous section of a passageway such as a sigmoid colon. In this case the skeletal member is delivered to and extends at least partially through and thereby at least partially bridges a tortuous section of passageway. The skeletal member in this case provides an overtube. A probe may then be advanced through the skeletal member, for example for visual examination of a section of colon. The inspection may be carried out as the device is being withdrawn from the colon.

In this case, a guide device similar to the guide device 1 of FIGS. 1 to 28 is advanced using a delivery member. The guide device comprises an evertable tube 3 having longitudinally extending outer and inner walls which are connected at each end to define an enclosed space, the inner wall defining a lumen 8, and a skeletal member 6 extending longitudinally in the enclosed space of the evertable tube 3. A delivery member which may be of similar form to a probe is inserted through the lumen 8 of the tube 3. The tube is inflated to grip the delivery member and the delivery member is advanced through the passageway with associated eversion of the tube and advancement of the skeletal member 6. The evertable tube 3 is then deflated such that the skeletal member bridges a section of the passageway.

After bridging of the section of the passageway the delivery member is withdrawn from the lumen 8 of the tube 3. The skeletal member thereby provides an overtube. A probe may then be inserted through the lumen 8 defined by the skeletal member 6 for further advancement as described above and/or for inspection or drawing back of the guide member and probe.

The invention is not limited to the embodiments hereinbefore described, with reference to the accompanying drawings, which may be varied in construction and detail.

1: A guide device for a probe, the device comprising: an evertable tube, the tube having longitudinally extending outer and inner walls which are connected at each end to define an enclosed inflation space therebetween, the inner wall defining a lumen and the tube being inflatable to grip a probe in the lumen such that the tube is evertable with advancement of a probe.

2: A guide device as claimed in claim 1 comprising a skeletal member extending longitudinally in the inflation space.

3: A guide device as claimed in claimed in claim 2 wherein the skeletal member comprises a tubular skeletal member.

4: A guide device as claimed in claim 2 wherein the skeletal member is at least partially corrugated.

5: A guide device as claimed in claim 2 wherein the skeletal member is slitted.

6: A guide device as claimed in claim 5 wherein the slit extends at least partially in a spiral.

7: A guide device for a probe, the device comprising an evertable tube with a lumen therethrough, the tube defining an inflation space and being inflatable to grip a probe in the lumen, and a skeletal member extending longitudinally of the expansion space of the tube.

8: A guide device for a probe, the device comprising an evertable tube with a lumen therethrough, the tube defining an inflation space and being inflatable to grip a probe in the lumen, and the tube being longitudinally extendable over a probe in the lumen for alignment of an end of the probe with an end of the tube.

9: A guide device for a probe, the device comprising an evertable tube with a lumen therethrough, the tube defining an inflation space and being inflatable to grip a probe in the lumen, and a stop to selectively prevent tube eversion.

10: A guide device for a probe, the device comprising, an evertable tube with a lumen therethrough, the tube being inflatable to grip a probe in the lumen such that the tube everts with advancement of the probe; the tube being advanceable over a probe in the lumen to align an end of the tube with an end of the probe.

11: A device as claimed in claim 10 wherein the outer wall of the tube is connected at each end to the inner wall of the tube to define an enclosed inflation space therebetween.

12: A device as claimed in claim 10 wherein the tube is longitudinally extendable from a shortened configuration to a lengthened configuration over a probe in the lumen to align an end of the tube with an end of the probe.

13: A device as claimed in claim 12 wherein in the shortened configuration a part of the tube is bunched.

14: A device as claimed in claim 13 wherein the bunched part of the tube is provided on the outer wall of the tube.

15: A device as claimed in claim 12 wherein at least part of the tube is inflatable to longitudinally extend the tube.

16: A device as claimed in claim 15 wherein the inflatable part of the tube is conically shaped.

17: A device as claimed in claim 12 wherein the device comprises stop means to selectively prevent longitudinal extension of the tube.

18: A device as claimed in claim 1 wherein the device comprises stop means to selectively prevent tube eversion.

19: A device as claimed in claim 17 wherein the stop means comprises a clamp engagable with the tube.

20: A device as claimed in claim 10 wherein the device comprises a skeletal member extending longitudinally of the tube.

21: A device as claimed in claim 20 wherein the tube has an inflation space and the skeletal member extends in the inflation space.

22: A guide device for a probe, the device comprising an evertable tube with a lumen therethrough, the tube being inflatable to grip a probe in the lumen such that the tube everts with advancement of the probe; and a skeletal member extending longitudinally of the tube.

23: A device as claimed in claim 21 wherein the skeletal member is tubular.

24: A device as claimed in claim 21 wherein the skeletal member is at least partially corrugated.

25: A device as claimed in claim 24 wherein the corrugation extends in a convoluted manner.

26: A device as claimed in claim 24 wherein the corrugation extends in a loop.

27: A device as claimed in claim 21 wherein the skeletal member has at least one slit extending at least partially along the skeletal member in a spiral.

28 (canceled)

29: A probe system comprising a probe, and a guide device for the probe as claimed in claim 1.

30: A method of advancing a probe through a passageway, the method comprising the steps of: providing a probe; providing a guide device for the probe, the guide device having a lumen therethrough; inserting the probe through the lumen of the guide device; inflating the guide device to grip the probe; advancing the probe through the passageway with an associated eversion of the guide device; advancing at least part of the guide device over the probe to align the leading end of the guide device with the leading end of the probe.

31: A method as claimed in claim 30 wherein the method comprises the step of deflating the guide device and the guide device is then advanced over the probe by pushing the guide device over the probe.

32: A method as claimed in claim 30 wherein at least part of the guide device is advanced over the probe by inflating at least part of the guide device.

33: A method as claimed in claim 32 wherein a leading end part of the guide device is inflated.

34: A method as claimed in claim 33 wherein part of the guide device is deflated before inflation of the leading end part.

35: A method as claimed in claim 30 wherein the method comprises the step of releasing a stop means before advancing at least part of the guide device over the probe.

36: A method as claimed in claim 30 wherein the probe is advanced through the passageway by pushing the probe through the passageway.

37: A method as claimed in claim 30 wherein the probe is maintained in a fixed position during the step of advancing at least part of the guide device over the probe.

38: A method as claimed in claim 30 wherein the method comprises the step of releasing a stop means before eversion of the guide device.

39: A method as claimed in claim 30 wherein at least some of the steps are repeated to advance the probe in an incremental manner through the passageway.

40: A method of advancing a probe through a passageway, the method comprising the steps of: providing a probe; providing a guide device for the probe, the guide device comprising an evertable tube having longitudinally extending outer and inner walls which are connected at each end to define an enclosed inflation space therebetween, the inner wall defining a lumen; inserting the probe through the lumen of the guide device; inflating the guide device to grip the probe; pushing the probe to advance the probe through the passageway which causes eversion of the guide device; moving at least part of the probe relative to the guide device to align the leading end of the probe with the leading end of the guide device.

41: A method as claimed in claim 40 wherein at least part of the guide device is advanced over the probe to align the leading end of the guide device with the leading end of the probe.

42: A method as claimed in claim 40 wherein the probe is retracted through the lumen to align the leading end of the guide device with the leading end of the probe.

43 (canceled)

44: A method of bridging a tortuous section of a passageway such as a sigmoid colon comprising the steps of: providing an evertable tube, the tube having longitudinally extending outer and inner walls which are connected at each end to define an enclosed space, the inner wall defining a lumen; providing a skeletal member extending longitudinally of the enclosed space of the evertable tube; providing a delivery member; inserting the delivery member through the lumen of the evertable tube; inflating the tube to grip the delivery member; advancing the delivery member through the passageway with associated eversion of the tube and advancement of the skeletal member; and deflating the evertable tube such that the skeletal member bridges a section of the passageway.

45: A method as claimed in claim 44 comprising withdrawing the delivery member after bridging of the section of the passageway.

46: A method as claimed in claim 45 comprising insertion of a probe in a lumen defined by the skeletal member.

47 (canceled)
